SEQUENCE LISTING (2 sequences)

SEQ ID NO: 1 - DNA, 22 bp, Artificial Sequence
  Mhyo-PCR-16S-rRNA Forward primer
gtagaaaggaggtgttccatcc

SEQ ID NO: 2 - DNA, 20 bp, Artificial Sequence
  Mhyo-PCR-16S-rRNA Reverse primer
acgctagctgtgtgcttaat

(19) United States
(12) Patent Application Publication
(10) Pub. No.: US 2022/0235313 A1
(43) Pub. Date: Jul. 28, 2022

(54) MYCOPLASMA MEDIA FORMULATIONS

(71) Applicant: ELANCO US, INC., Greenfield, IN (US)

(72) Inventors: Arvind KUMAR, Greenfield, IN (US); Dharanesh Mahimapura GANGAIAH, Greenfield, IN (US)

(21) Appl. No.: 17/617,481

(22) PCT Filed: Jun. 5, 2020
(86) PCT No.: PCT/US2020/036297

§ 371 Date: Dec. 8, 2021

Related U.S. Application Data
(60) Provisional application No. 62/859,294, filed on Jun. 10, 2019.

(51) Int. Cl. C12N 1/20

(52) U.S. Cl. CPC C12N 1/20; C12N 2500/38; C12N 2500/32

(57) ABSTRACT

The present invention relates to media formulations free of swine serum and animal (primarily bovine brain and spinal cord) origin ingredients for Mycoplasma growth. Media formulations are rationally designed to preserve Mycoplasma antigenicity. Mycoplasma grown in these media formulations are useful in vaccines, particularly multivalent swine vaccines.

### FIELD OF THE INVENTION

The present invention relates to media formulations free of swine serum and with minimal or no animal origin ingredients. The media formulations are useful for the growth of Mycoplasma species, in particular Mycoplasma hyopneumoniae. The media formulations are rationally designed to optimize Mycoplasma growth while maintaining antigenic gene expression. Mycoplasma grown in the disclosed media formulations are suitable for use in swine vaccines.

### BACKGROUND OF THE INVENTION

Mycoplasma are small gram-negative bacteria which lack a cell wall, are generally nonmotile, and are often parasitic or pathogenic to mammals, birds, reptiles, amphibians, fish, insects, and even plants. A large number of Mycoplasma species are classified within the family Mycoplasmataceae. Mycoplasmas may be commensal bacteria and are often found in association with mucous membranes of mammals. More than one Mycoplasma species may colonize a particular mucosal surface. Mycoplasmas have been implicated as causative agents of various disease states, particularly in immunocompromised organisms. In some cases, Mycoplasma pathogenicity may be associated with the presence of viruses or other bacteria. Mycoplasmas may also act as secondary infectious agents.

To control, reduce or prevent Mycoplasma-related diseases, effective vaccines are desired. Cultures of Mycoplasma are needed to produce antigens for such vaccines, but the very nature of Mycoplasmas present difficulties. Mycoplasmas are the smallest self-replicating non-viral organisms and they contain correspondingly small genomes, estimated at less than about a thousand total genes. This restricted genome lacks many enzymes required for the production of essential nutrients, so Mycoplasma are dependent on host cell factors or cell culture supplements for growth. For example, Mycoplasma often require external sources of guanine and cytosine nucleosides and cholesterol or other lipids. Thus, Mycoplasma necessarily takes on characteristics of its environment, which alters antigen expression and immunogenicity, and vaccines containing Mycoplasma vary in effectiveness depending on the process and supplements used for growing the Mycoplasma.

Multiple Mycoplasma species have been implicated in diseases of swine. M. hyosynoviae is thought to cause arthritis in pigs, M. suis can result in anemia, and M. hyorhinitis may contribute to fibrinous polyserositis especially in young pigs. M. hyopneumoniae (“Mhp”) causes enzootic pneumonia and is a factor in the porcine respiratory disease complex (PRDC) along with Porcine Reproductive and Respiratory Syndrome (PRRS) virus and influenza virus.

Polyvalent vaccines are often desirable, where Mycoplasma antigens are combined with one or more other bacteria or viruses, such as for example PRRS virus, influenza virus, porcine parvovirus, African Swine Fever virus, and/or porcine circovirus-2 (PCV-2). However, while Mycoplasma is most effective as an antigen when grown in swine serum as a source of cholesterol, etc., the serum contains antibodies which could interfere with immunization against other pathogens, particularly PCV-2. To reduce or eliminate anti-PCV2 antibodies, others have removed the antibodies by Protein A/G columns (see e.g. U.S. Pat. No. 9,120,859), but this is laborious and costly. Low serum culture systems have been developed (see e.g. U.S. Pat. No. 9,273,281), but these may contain contaminating eukaryotic cell factors and the Mycoplasma thus cultured may not have optimal immunogenicity.

What is needed is an improved method of culturing Mycoplasma wherein growth is optimized but contaminants are reduced or eliminated, and immunogenicity is preserved. For commercial purposes, the method should be cost-efficient and easily implemented, and undesirable contaminants should be limited. Disclosed herein is a rationally designed method of culturing Mycoplasma, media formulations for performing the method, and vaccines containing Mycoplasma preparing using the method.

### SUMMARY OF THE INVENTION

The present invention provides a composition comprising choline chloride, niacinamide, nicotinic acid, L-methionine, L-cysteine, putrescine dihydrochloride, thiamine pyrophosphate, sodium L-ascorbate, spermine, pyridoxal 5′-phosphate monohydrate, tetrahydrofolic acid, 3′-dephosphocoenzyme, and riboflavin. The composition may be used as a Mycoplasma growth supplement (“MGS”). The Mycoplasma grown using the MGS may be M. hyosynoviae; M. suis; M. hyorhinitis; or M. hyopneumoniae (“Mhp”). The Mycoplasma grown using the MGS may be M. hyopneumoniae. The Mycoplasma grown using the MGS may be any Mycoplasma capable of growth in or on a porcine animal, tissue, or cell. The components of the MGS may be present in a complete growth medium at final concentrations of: about 0.5 mg/L choline chloride; about 0.025 mg/L niacinamide; about 0.025 mg/L nicotinic acid; about 0.1 mM L-methionine; about 1.5 mM L-cysteine; about 0.1 mM putrescine dihydrochloride; about 0.01 mg/L thiamine pyrophosphate; about 0.284 mM sodium L-ascorbate; about 0.1 mM spermine; about 0.025 mg/L pyridoxal 5′-phosphate monohydrate; about 0.05 mg/L tetrahydrofolic acid; about 0.025 mg/L 3′-dephosphocoenzyme A; and about 0.01 mg/L riboflavin.

The present invention provides a method of culturing Mycoplasma, comprising placing Mycoplasma in a media comprising basal medium, horse serum; and a Mycoplasma growth supplement. The basal medium is selected from Frey's medium and porcine brain heart infusion (p-BHI) medium. The MGS comprises choline chloride, niacinamide, nicotinic acid, L-methionine, L-cysteine, putrescine dihydrochloride, thiamine pyrophosphate, sodium L-ascorbate, spermine, pyridoxal 5′-phosphate monohydrate, tetrahydrofolic acid, 3′-dephosphocoenzyme, and riboflavin. The components of the MGS may be present in a complete growth medium at final concentrations of: about 0.5 mg/L choline chloride; about 0.025 mg/L niacinamide; about 0.025 mg/L nicotinic acid; about 0.1 mM L-methionine; about 1.5 mM L-cysteine; about 0.1 mM putrescine dihydrochloride; about 0.01 mg/L thiamine pyrophosphate; about 0.284 mM sodium L-ascorbate; about 0.1 mM spermine; about 0.025 mg/L pyridoxal 5′-phosphate monohydrate; about 0.05 mg/L tetrahydrofolic acid; about 0.025 mg/L 3′-dephosphocoenzyme A; and about 0.01 mg/L riboflavin.

The present invention provides a method of culturing Mycoplasma, wherein the Mycoplasma grown using the method may be any Mycoplasma capable of growth in or on a porcine animal, tissue, or cell. The Mycoplasma grown using the method may be M. hyosynoviae; M. suis; M. hyorhinitis; or M. hyopneumoniae (“Mhp”). The Mycoplasma grown using the method may be M. hyopneumoniae.

The present invention provides a method of culturing Mycoplasma, comprising placing Mycoplasma in a media comprising basal medium, horse serum; and a Mycoplasma growth supplement. The horse serum may be present in the complete medium at about 2.5% to about 10% v/v. The horse serum may be present in the complete medium at about 5% to about 10% v/v. The horse serum may be present in the complete medium at about 10% v/v. The horse serum may be present in the complete medium at about 2%, 2.5%, 3%, 4%, 5%, 6%, 7%, 8%, 9%, or 10%.

The present invention provides a method of culturing Mycoplasma, comprising placing Mycoplasma in a media comprising basal medium, horse serum; and a Mycoplasma growth supplement, and culturing the Mycoplasma at 37° C. for 3-15 days. The present invention provides a method of culturing Mycoplasma, comprising placing Mycoplasma in a media comprising basal medium, horse serum; and a Mycoplasma growth supplement, and culturing the Mycoplasma at 37° C. for 3, 4, 5, 6, 7, 8, 9, 10, 11, 12, 13, 14, or 15 days. The Mycoplasma culturing may also include orbital shaking.

The present invention provides a method of preparing an immunogenic composition comprising placing Mycoplasma in a media comprising basal medium, horse serum; and a Mycoplasma growth supplement; incubating the Mycoplasma at 37° C.; and inactivating the Mycoplasma. The basal medium is selected from Frey's medium and porcine brain heart infusion (p-BHI) medium. The MGS comprises choline chloride, niacinamide, nicotinic acid, L-methionine, L-cysteine, putrescine dihydrochloride, thiamine pyrophosphate, sodium L-ascorbate, spermine, pyridoxal 5′-phosphate monohydrate, tetrahydrofolic acid, 3′-dephosphocoenzyme, and riboflavin. The horse serum may be present in the complete medium at about 2.5% to about 10% v/v. The Mycoplasma to be included in the immunogenic composition may be any Mycoplasma capable of growth in or on a porcine animal, tissue, or cell. The Mycoplasma to be included in the immunogenic composition may be M. hyosynoviae; M. suis; M. hyorhinitis; or M. hyopneumoniae (“Mhp”). The Mycoplasma may be inactivated with 2-bromoethylamine.

The present invention provides for use of an MGS in the manufacture of a medicament for preventing, reducing, or ameliorating diseases caused by Mycoplasma. The MGS comprises choline chloride, niacinamide, nicotinic acid, L-methionine, L-cysteine, putrescine dihydrochloride, thiamine pyrophosphate, sodium L-ascorbate, spermine, pyridoxal 5′-phosphate monohydrate, tetrahydrofolic acid, 3′-dephosphocoenzyme, and riboflavin. The medicament may be an immunogenic composition or a vaccine. The medicament may be useful for treatment of human and non-human animals, including swine.

### BRIEF DESCRIPTION OF THE DRAWINGS

FIG. 1. Full-scale CCU assay results of three-day cultures of Mhp in the indicated media formulations. The data represent the mean of three experimental replicates with the exception that the three formulations containing Acutone were only included in one experiment.

FIG. 2. Growth of Mhp in six different media formulations in a large-scale fermenter system, as determined by a full-scale CCU assay. The Day 0 CCU levels are estimated on the inoculum.

FIG. 3. Lung lesion scores of pigs vaccinated with Mhp grown in experimental media formulations and then challenged with virulent Mhp. Gross lung lesion scoring is performed using the cranio-ventral pulmonary consolidation (CVPC) method and expressed as total lung lesion percentage, defined as the sum of the lung lesion percent in the separate seven lobes (right apical, right cardiac, right caudal, left caudal, left cardiac, left apical and intermediate) of lungs multiplied by the approximate volume of each lung lobe contributed to the entire lung. The commercial vaccine FOSTERA (Zoetis Animal Health) is presented as a positive control.

FIG. 4. PCV-2 neutralizing antibody titers in serum of vaccinated pigs as measure by ELISA. ELISA was performed using the INGEZIM CIRCO IgG ELISA kit, following manufacturer's recommendations (Professional Veterinary Service, Inc.).

### DETAILED DESCRIPTION OF THE INVENTION

As used in the following discussion, the terms “a” or “an” should be understood to encompass one or more, unless otherwise specified.

As used herein, “Mycoplasma” refers to any species classified within the family Mycoplasmataceae. The term may mean a single organism or a culture containing a plurality of organisms. Particularly included in this definition is M. hyosynoviae, M. suis, M. hyorhinitis, and M. hyopneumoniae (“Mhp”). As used herein, “inactivated” Mycoplasma mean organisms which can no longer replicate in a host or in culture. Inactivated organisms are considered to be killed or dead. Inactivation can be accomplished by a variety of methods, including but not limited to chemical alteration of proteins, to chemical or physical alterations in the structure of a cell, or to chemical or physical alterations in nucleic acids.

As used herein, “Mycoplasma growth supplement” (“MGS”) means a rationally-designed composition of components identified to be important for Mycoplasma growth. Identification of the components is based on a genomic analysis of Mhp. MGS is typically prepared as a concentrated solution which is added to a basal media formulation during the preparation of complete media. The MGS disclosed herein comprises choline chloride, niacinamide, nicotinic acid, L-methionine, L-cysteine, putrescine dihydrochloride, thiamine pyrophosphate, sodium L-ascorbate, spermine, pyridoxal 5′-phosphate monohydrate, tetrahydrofolic acid, 3′-dephosphocoenzyme, and riboflavin. A person of skill in the art would recognize some salt forms of these components may be interchangeable.

As used herein, “complete media” refers to a culture medium which contains the necessary organic factors for the growth of an indicated organism or cell. For Mhp, complete media at least comprises basal media, serum, and MGS. “Final concentration” means the concentration of an indicated component in a complete media.

As used herein, “Frey” medium and “porcine brain heart infusion” (“p-BHI”) medium mean the formulations identified in Tables 11 and 12.

As used herein, an “immunogenic composition” is a composition that elicits an immune response when administered to an animal. An immunogenic composition comprises at least one antigen and at least one pharmaceutically-acceptable excipient. The antigen can be a whole virus, bacterium, or other pathogen, either live or inactivated. The antigen can also be isolated, purified, or partially purified antigenic molecule from a virus, bacterium, or other pathogen. The antigen can be a polypeptide, a polysaccharide, a nucleic acid, or a lipid, or any combinations thereof.

As used herein, a “vaccine” is an immunogenic composition which confers protection from, resistance to, prevention of, or reduction for a disease symptom when administered to an animal, wherein said symptom is caused by a pathogenic organism, for example a bacterium, more particularly a Mycoplasma.

As used herein, the term “porcine” and “swine” refers to pigs, any of the animals in the genus Sus within the even-toed ungulate family Suidae.

The term “about” will be understood by persons of ordinary skill in the art and will vary to some extent depending on the context in which it is used. As used herein, “about” is meant to encompass variations of ±10% of the value with which the term is associated.

As used herein, the terms “treating”, “to treat”, or “treatment”, include restraining, slowing, stopping, reducing, ameliorating, or reversing the progression or severity of an existing symptom, disorder, condition, or disease. A treatment may be applied prophylactically or therapeutically.

The following experimental examples are illustrative of methods for culturing Mycoplasma, including materials useful therein and uses for the Mycoplasma so cultured. It will be appreciated that other embodiments and uses will be apparent to those skilled in the art and that the invention is not limited to these specific illustrative examples or preferred embodiments.

The purpose of the studies presented herein is to determine if M. hyopneumoniae could be successfully grown in medium without porcine serum and without bovine brain, heart infusion (or any animal ingredients, if possible). The most successful formulations would then be used to produce material for efficacy studies in animals. The major response variable for evaluating success of the new media formulations was viable cell count estimated by color-changing unit (CCU) assay. Proteomics analysis of the cells grown in the most promising formulations is performed to determine if the absence of porcine serum significantly altered the protein expression profile of the cells, which could decrease the antigenic properties of the cells and diminish the immunogenic effect of the vaccine product.

All percentages given are volume per volume (v/v) unless otherwise stated.

### Example 1

The objective of this study is to confirm the identity of a M. hyopneumoniae (Mhp) strain and establish working stocks for further experimentation.

A North American strain of Mhp designated GL713 (the active ingredient in PNEUMOSTAR MYCO) is used to establish working stocks. For Examples 1, 4, and 5, a 0.5 mL aliquot of an X+4 passage of GL713 is used to inoculate 25 mL Friis media (Teknova) containing 10% swine serum (HyClone cat. #SH30908.04) in a 125 mL baffled, non-vented polycarbonate flask. After 3 days at 37° C. with orbital agitation (100 rpm), 12.5 mL of the culture is used to inoculate 100 mL Friis medium containing 10% swine serum in a 500 mL flask. After 3 days, 100 mL Friis medium containing 20% glycerol with 10% swine serum is added to the culture and mixed. The mixture is aliquoted (1 mL) and frozen at −80° C. to establish X+5 working seeds. During the course of the presented studies, Mhp cultures are passed every 3-4 days to fresh Friis medium containing 10% swine serum to maintain a source of inocula at passage X+6.

To confirm the identity of the GL713 seeds, genomic DNA is isolated using the phenol-chloroform method. The quality and quantity of the isolated genomic is assessed using NANODROP (ThermoScientific) and agarose gel electrophoresis. Agarose gel electrophoresis shows the presence of genomic DNA running above 8 kb. NANODROP analysis shows a genomic DNA yield of 77.3 ng/uL.

The genomic DNA is analyzed by polymerase chain reaction (PCR) methods to confirm the identity of the 16S rRNA gene. Briefly, a 50 uL PCR reaction mix is prepared using 25 uL of GREENTAQ HOT START GREEN PCR master mix (ThermoScientific), 1 uL of forward primer (5′ GTAGAAAGGAGGTGTTCCATCC 3′; SEQ ID NO: 1), 1 uL of reverse primer (5′ ACGCTAGCTGTGTGCTTAAT 3′; SEQ ID NO: 2), 20.0 uL of H₂O and 3 uL of isolated genomic DNA. For PCR amplification, an initial denaturation temperature of 95° C. is performed for 3 minutes, and then 35 cycles of: a denaturation temperature of 95° C. for 30 seconds, an annealing temperature of 60° C. for 30 seconds, and an extension temperature of 72° C. for 1 minute. A final extension step is conducted at 72° C. for 10 minutes. The PCR products were purified using QIAQUICK PCR Purification Kit (Qiagen). The quality and quantity of the PCR products were assessed using NANODROP and agarose gel electrophoresis. The results of PCR amplification showed a PCR product of approximately 1600 bp, correlating to the expected size is 1503 bp.

The purified PCR products are diluted and mixed with primers to generate sequencing premixed samples (GenScript). The PCR products are sequenced from both ends (i.e. using both forward and reverse primers). Following sequencing, the results are analyzed using Basic Local Alignment Search Tool, and the GL713 16S rRNA sequence is confirmed to be 99% identical to M. hyopneumoniae 232 (GenBank Accession no. AE017332).

### Example 2

To prepare stocks of GL713 for use in these studies, a 1 mL aliquot of an X+1 passage of GL713 is used to inoculate 50 mL Friis media containing 10% swine serum in a 250 mL baffled, non-vented polycarbonate flask. After 4 days at 37° C. with orbital agitation (100 rpm), 15 mL fresh Friis base medium containing 20% glycerol but no swine serum is added to the culture and mixed. Aliquots of 1 mL each are frozen at −80° C. to establish X+2 pre-pre-master seeds. One aliquot is used to inoculate 3×500 mL baffled flasks each containing 100 mL of Friis containing 10% swine serum. The culture is incubated for 5 days at 37° C. with 100 rpm orbital shaking. On day 5, the 3 flasks are combined to yield a 300 mL culture. The pre-master seed at X+3 is prepared by combining the 300 mL culture with 300 mL fresh Friis base medium containing 20% glycerol (no swine serum) and storing in 1.25 mL aliquots at −80° C. The pre-master seed is tested for viability using a full-scale CCU assay and for sterility using blood agar plates incubated under both aerobic and anaerobic conditions and at both room temperature and 37° C. During the course of the presented studies, Mhp cultures are passed every 3-4 days to fresh Friis medium containing 10% swine serum to maintain a source of inocula at passage X+4.

Whole-genome sequencing of the X+2 pre-pre-master seed is performed to further confirm the identity of GL713 and to facilitate pathway analysis to identify potential metabolic/nutritional capabilities or deficiencies of Mhp.

High molecular weight genomic DNA is isolated using phenol-chloroform method. The quality and quantity of the isolated genomic is assessed using NANODROP and agarose gel electrophoresis. Agarose gel electrophoresis shows the presence of genomic DNA running above 8 kb, and NANODROP analysis shows a genomic DNA yield of 377.6 ng/uL. The isolated DNA is sequenced by ACGT, Inc. (Wheeling, Ill., USA). Following sequencing, the contigs are annotated and analyzed for identity by BLAST.

Sequencing of GL713 yields 38,410, 897 raw reads by paired end sequencing and 3,102,689 reads by mate pair sequencing. The final assembly reveals 5 contigs. The estimated genome size of GL713 is 862,838 bp. BLAST analysis shows the genomic sequence of GL713 is 99% identical to M. hyopneumoniae 232.

### Example 3

The objective of this study is to identify the key metabolic requirements of Mhp through metabolic pathway analysis. The genomic sequences of GL713 from Example 2 and of M. hyopneumoniae 232 (GenBank Accession no. AE017332) are analyzed using the following four approaches:
bioinformatically, pathways are identified by comparing the Mhp genomes to Escherichia coli and Coxiella burnetii genomes;
the M. hyopneumoniae GL713 and 232 genomes are manually scanned for the existing metabolic pathways;
pathway information is collected from the published literature (both in silico and experimental studies); and
predicted pathway information from other Mycoplasma species, particularly human Mycoplasmas, are compared to the Mhp genomic sequences.

Based on this analysis, Mhp does not appear to contain pathways for the synthesis of amino acids, although there are several amino acid and peptide transporters in the membrane. Thus, Mhp absolutely requires external sources of amino acids and/or peptides.

Mhp has a limited capacity to synthesize lipids and has no discernable fatty acid biosynthetic pathways. Thus, Mhp absolutely requires external sources of fatty acids, glycerol, glycerophosphodiesters and choline. Cholesterol is likely required for membrane stability and may be adsorbed onto the cell surface, but no pathways or transporters to utilize cholesterol are identified.

Mhp has no ability to de novo synthesize purines and pyrimidines for DNA and RNA construction. However, Mhp does have a pathway to utilize L-ascorbate, which feeds into the pentose phosphate pathway, and thus it can make phospho-ribose precursors for DNA and RNA synthesis. Thus, Mhp requires external sources of guanine, adenine, cytidine, uracil, and thymidine, and also an external source of either L-ascorbate or ribose.

Mhp has complete pathways for the utilization of glucose; and glucose appears to be the preferred carbon source. Glucose enters the glycolytic pathway to generate pyruvate; and pyruvate either enters the acetate pathway to generate acetate as the end product or the lactate pathway to generate lactate as the end product. Both glycolytic and acetate pathways generate ATP and the lactate pathway regenerates NAD, which is required for ATP synthesis. The CCU assay is based on conversion of glucose to lactic acid, which leads to a pH change and consequently color change of phenol red in the media. Thus, Mhp requires glucose in the media, although alternative carbon sources—mannitol, fructose, glycerol, mannose, L-ascorbate—are possible as Mhp has transporters for all these carbon sources in the membrane. One glucose source may be serum, which contains 1-2 mM glucose. However, Mhp has no functional TCA cycle, and thus it does not need a high level of glucose like E. coli due to lack of the TCA cycle and other glucose intensive pathways.

Of all the Mycoplasma species so characterized, only Mhp appears to contain a myo-inositol catabolic pathway. Thus, in Mhp myo-inositol could serve as a carbon source and a precursor for CoA. A transporter for myo-inositol is also present in the Mhp membrane.

Among cofactors necessary for proper metabolic activity, Mhp appears to have no means to synthesize, and thus requires external sources of, tetrahydrofolate, 4-phosphopantothene, riboflavine, pyridoxal-5-phosphate, and thiamine pyrophosphate. Similarly, Mhp appears to have no means to synthesize, and thus requires external sources of, the polyamines spermine and putrescine, although membrane-integrated transporters for these molecules are present in Mhp. Other potential nutritional requirements include L-cysteine and methionine.

Based on the collated information obtained from the pathway analysis, a supplement called Mycoplasma Growth Supplement (MGS) is designed. The ingredients and their concentration in this supplement are described in Table 1. The collated pathway information can also be used to select base media and other ingredients, which are experimentally tested in the following Examples.

TABLE 1
Rationally-designed Mycoplasma Growth Supplement (MGS).

| | Final Concentration | | | |
|---|---|---|---|---|
| Component | in Medium¹ | | Sigma cat. # | |
| Choline chloride | 0.5 | mg/L | C7527 | (100 g) |
| Niacinamide | 0.025 | mg/L | N5535 | (100 g) |
| Nicotinic acid | 0.025 | mg/L | N0761 | (100 g) |
| L-methionine | 0.1 | mM | M5308 | (100 g) |
| L-cysteine | 1.5 | mM | C7352 | (100 g) |
| Putrescine dihydrochloride | 0.1 | mM | P5780 | (5 g) |
| Thiamine pyrophosphate | 0.01 | mg/L | C8754 | (5 g) |
| Sodium L-ascorbate | 0.284 | mM | A4034 | (100 g) |
| Spermine | 0.1 | mM | S3256 | (5 g) |
| Pyridoxal 5′-phosphate | 0.025 | mg/L | 82780 | (1 g) |
| monohydrate | | | | |
| Tetrahydrofolic acid | 0.05 | mg/L | T3125 | (100 mg) |
| 3′-dephosphocoenzyme A | 0.025 | mg/L | D3385 | (25 mg) |
| Riboflavin | 0.01 | mg/L | R9504 | (25 g) |

¹MGS is typically prepared as a 100X stock, aliquoted and frozen.

### Example 4

The objective of this study is to evaluate the growth of Mhp GL713 in response to the following variables using a truncated CCU assay:

1. Base medium: Vegetone Infusion Broth (no animal ingredients; Sigma, cat. #41960), AF Friis (no animal ingredients; Becton Dickinson experimental formulation, batch #CRD17082), AF PPLO (no animal ingredients; Becton Dickinson experimental formulation, batch #CRD17079), Acutone (no animal ingredients, Neogen/Acumedia, cat. #7742A), Friis (contains bovine brain heart infusion; Teknova, cat. #F0485), Porcine Brain Heart Infusion (porcine BHI; Becton Dickinson, cat. #BD256120), and Frey (contains pancreatic digest; Becton Dickinson, cat. #212346). These media are selected to provide basic nutritional requirements of Mhp based on the information from pathway analysis. Animal free base media are included to facilitate global registration of a vaccine product.

2. Serum source: swine and horse. Although other serum sources such as chicken, turkey and rabbit have been used previously for Mycoplasma growth, cost analysis indicates that these serum sources would increase the cost of growth media and hence were not tested. Horse serum (Sigma, cat. #H1138-500 mL) is comparable in cost to swine serum.

3. Swine serum level: 1, 5, and 10%. The current common media formulation, Friis Mycoplasma Base Medium (Teknova), uses 10% swine serum for growth. If Mhp does not grow in the absence of swine serum, reduction of swine serum in the media might ease downstream removal of contaminating antibodies in an Mhp antigenic preparation.

4. Mycoplasma Growth Supplement (MGS): 1× and 2.5×. (Table 1).

5. Myoinositol: 1×, 2×, and 10× (SigmaAldrich). The metabolic pathway analysis reveals Mhp contains a complete pathway for myoinositiol catabolism, suggesting that myoinositol might serve as a source of energy for Mhp growth.

6. Select Phytone: Based on the pathway analysis, Mhp cannot synthesize amino acids and contains genes encoding membrane transporters for amino acids and peptides. Since DIFCO SELECT PHYTONE UF (Becton Dickinson, cat. #210931) is animal origin-free and is a rich source of amino acids and peptides, addition of this nutrient might enhance Mhp growth.

7. Yeast extract: Yeast extract (BD Biosciences) is a rich source of peptides and amino acids, which Mhp absolutely requires for growth.

8. 7.6 and 8.0. In Wodke et al. (Molecular Systems Biology 9: 653, 2013) the authors showed that Mhp utilized more glucose and accumulated more protein at pH 8.0 compared to pH 7.5.

9. Egg yolk extract: Egg yolk is a rich source of cholesterol, fatty acids and amino acids. Previously, egg yolk extract (SigmaAldrich) has been successfully used to grow Mycoplasma (Sasaki et al., Microbiol Immunol. 29(6): 499-507, 1985).

10. Glucose: 1, 2, 3, and 4 g/L. In general, serum contains around 4 g/L of glucose and, based on the information from pathway analysis, glucose appears to be a key carbon source for Mhp. Therefore, the effect of glucose on Mhp growth is tested, especially when serum was not included in the media formulation.

11. Glycerol: Mhp contains pathway for metabolism of glycerol, which can serve as both a carbon source and a precursor for lipid biosynthesis. effect of glycerol on Mhp growth is tested.

To prepare the inoculum for these experiments (Examples 4 and 5), 100 mL of Friis containing 10% swine serum is inoculated in a 500 mL baffled flask with 2×1 mL frozen X+2 stock. Mhp are allowed to grow for three days and then growth is assessed using a truncated CCU assay, as a full CCU assay is very labor and space intensive. Briefly, a test sample is serially diluted 10-fold, vortex mixed and incubated for 3 days at 37° C. A minimum of 2 uninoculated tubes are included as negative control. Growth is indicated by pH shift, resulting in color change from red to yellow of phenol red in the media. Mhp growth is correlated with accumulation of lactic acid, which causes the pH shift. The endpoint titer is indicated by highest dilution (last) showing Mhp growth.

Nine rounds of testing media formulations with the different variables are performed. CCU assay results are obtained 3 days after preparation; thus, in some instances, the next experiment is set up before the results of the previous experiment are known. The cultures are also subjected to sterility (i.e contamination) testing on blood agar at the end of day 3 of culture.

TABLE 2
Round 1 testing of media variables.

| Formulation | Growth |
|---|---|
| Friis + 10% Swine Serum at pH 7.6 | Control |
| Friis + 10% Swine Serum at pH 8.0 | Similar to control |
| Vegitone Infusion Broth + Select Phytone | Poor/no growth |
| (1%) + 10% Swine Serum | |
| Vegitone Infusion Broth + Select Phytone | Poor/no growth |
| (5%) + 10% Swine Serum | |
| Vegitone Infusion Broth + 10% Swine Serum | Poor/no growth |
| Friis + 10% Horse Serum | < control |
| Friis + 10% Swine Serum + MGS (1X) | > control |
| Vegitone Infusion Broth + Select Phytone | Poor/no growth |
| (1%) + MGS (1X) | |
| Friis + MGS (1X) | < control |
| Vegitone Infusion Broth + Select Phytone | Poor/no growth |
| (1%) + MGS (2X) | |

Conclusions that may be drawn from this round of variable testing are that adjusting the pH of the media to 8.0 does not enhance Mhp growth compared to pH 7.6; Vegetone Infusion broth and Select Phytone do not support Mhp growth; horse serum appears to substitute for swine serum; and MGS enhances Mhp growth.

TABLE 3
Round 2 testing of media variables.

| Formulation | Growth |
|---|---|
| Friis + 10% Swine Serum at pH 7.6 | Control |
| Friis + 10% Horse Serum at pH 7.6 | < control |
| Friis + 10% horse serum + MGS (1X) + | ≥ control |
| myo-inositol (1X) | |
| Friis + MGS (1X) | Similar to control |
| Friis + MGS (1X) + myo-inositol (1X) | < control |
| Friis + MGS (2.5X) + myo-inositol (2.5X) | < control |
| Friis + MGS (1X) + myo-inositol (10X) | < control |
| Acutone + yeast extract (to 6 g/L) + Friis-equivalent | Poor/no growth |
| supplement + MGS (1X) + myo-inositol (1X) | |
| Acutone + egg yolk extract (10%) + MGS | Poor/no growth |
| (1X) + myo-inositol (1X) | |
| Acutone + 10% swine serum | > control |

Conclusions that may be drawn from this round of variable testing are that myo-inositol does not appear to enhance Mhp growth, and that Acutone (no animal ingredients) with swine serum supports higher growth than the control. Results from this round also confirm that horse serum can substitute for swine serum and that MGS enhances Mhp growth.

TABLE 4
Round 3 testing of media variables.

| Formulation | Growth |
|---|---|
| Friis + 10% Swine Serum at pH 7.6 | Control |
| Friis + MGS (1X) + Myo-Inositol (1X) | ≤ control |
| Acutone + MGS (1X) + Myo-Inositol (1X) | No growth |
| Acutone + MGS (1X) + Myo-Inositol (1X) + Glucose (1 g/L) | No growth |
| Acutone + MGS (1X) + Myo-Inositol (1X) + Glucose (2 g/L) | No growth |
| Acutone + MGS (1X) + Myo-Inositol (1X) + Glucose (3 g/L) | No growth |
| Acutone + MGS (1X) + Myo-Inositol (1X) + Glucose (4 g/L) | No growth |
| Acutone + MGS (1X) + Myo-Inositol (10X) + Glucose (2 g/L) | No growth |
| Acutone + Egg Yolk Extract (10%) + MGS | No growth |
| (1X) + Myo-Inositol (1X) + Glucose (2 g/L) | |

Conclusions that may be drawn from this round of variable testing are that addition of glucose does not appear to enhance Mhp growth, and that, although MGS enhances growth with Fris base medium, it does not enhance growth with Acutone base medium.

TABLE 5
Round 4 testing of media variables.

| Formulation | Growth |
|---|---|
| Friis + 10% Swine Serum at pH 7.6 | Control |
| Friis only | poor growth |
| Friis + MGS + Myo-Inositol (10X) | ≤ control |
| Friis + MGS + Myo-Inositol (100 mg/L) | ≤ control |
| Acutone + Swine Serum | Similar to control |
| Acutone + Horse Serum | Poor/no growth |
| Acutone + Horse Serum + MGS + | Poor/no growth |
| myo-inositol (100 mg/L) | |
| Acutone + Glycerol (1%) + MGS + | Poor/no growth |
| Myo-inositol (100 mg/L) | |

A conclusion that may be drawn from this round of variable testing is that increasing myo-inositol concentrations do not appear to enhance Mhp growth. While Mhp grows better in Acutone with swine serum than in control medium, in the absence of serum or in the presence of horse serum Acutone does not support Mhp growth.

TABLE 6
Round 5 testing of media variables.

| Formulation | Growth |
|---|---|
| Friis + 10% Swine Serum at pH 7.6 | Control |
| Friis only | < control |
| Friis + MGS | ≤ control |
| Friis + Swine Serum + MGS | Similar to control |
| Friis + Horse Serum | ≤ control |
| Friis + Horse Serum + MGS | ≥ control |
| Acutone | No growth |
| Acutone + MGS | No growth |
| Acutone + Swine Serum | Similar to control |
| Acutone + Swine Serum + MGS | Similar to control |
| Acutone + Horse Serum | Poor growth |
| Acutone + Horse Serum + MGS | Poor growth |

Conclusions that may be drawn from this round of variable testing are that MGS enhances growth with Fris base medium but not with Acutone base medium, and that Acutone does not support Mhp growth in the absence of swine serum.

TABLE 7
Round 6 testing of media variables.

| Formulation | Growth |
|---|---|
| Friis + 10% Swine Serum at pH 7.6 | Control |
| Porcine BHI | Poor growth |
| Porcine BHI + MGS | Poor growth |
| Porcine BHI + Swine Serum | Similar to control |
| Porcine BHI + Swine | ≤ control |
| Serum + MGS | |
| Porcine BHI + Horse Serum | Similar to control |
| Porcine BHI + Horse | Poor growth |
| Serum + MGS | |

A conclusion that may be drawn from this round of variable testing is that Porcine BHI with swine serum or horse serum behaves very similar to Friis medium in supporting Mhp growth.

TABLE 8
Round 7 testing of media variables.

| Formulation | Growth |
|---|---|
| Friis + 10% Swine Serum at pH 7.6 | Control |
| Friis + MGS | ≤ control |
| Friis + 10% Swine Serum + MGS | Similar to control |
| Friis + 1% Swine Serum + MGS | ≤ control |
| Friis + 5% Swine Serum + MGS | Similar to control |
| Friis + 10% Horse Serum + MGS | ≤ control |
| Acutone + 10% Swine Serum | Similar to control |
| Acutone + 10% Swine Serum + MGS | > control |
| Acutone + 1% Swine Serum + MGS | Poor growth |
| Acutone + 5% Swine Serum + | < control |
| Porcine BHI + 10% Swine Serum | Similar to control |
| Porcine BHI + 10% Swine | < control |
| Serum + MGS | |
| Porcine BHI + 1% Swine Serum + MGS | Poor growth |
| Porcine BHI + 5% Swine Serum + MGS | < control |

A conclusion that may be drawn from this round of variable testing is that reducing the swine serum percentage to 1% might hamper Mhp growth, especially with Acutone and Porcine BHI. Another conclusion that may be drawn from this round of variable testing is that reducing the swine serum percentage to 5% behaves very similar to control with Friis base medium for supporting Mhp growth, but 5% swine serum does not support Mhp growth with Acutone and Porcine BHI. Note this round was performed with MGS that was frozen and thawed couple times, explaining the inefficient effect of MGS on Mhp growth.

TABLE 9
Round 8 testing of media variables.

| Formulation | Growth |
|---|---|
| Friis + 10% Swine Serum at pH 7.6 | Control |
| AF Friis + MGS | Poor/no growth |
| AF Friis + 10% Swine Serum | Similar to control |
| AF Friis + 10% Horse Serum + MGS | Poor growth |
| AF PPLO + MGS | no growth |
| AF PPLO + 10% Swine Serum | < control |
| AF PPLO + 10% Horse | Poor growth |
| Serum + MGS | |

A conclusion that may be drawn from this round of variable testing is that AF Friis medium supports Mhp growth similar to control medium in the presence of swine serum, but unlike regular Friis medium, AF Friis and AF PPLO media do not support Mhp growth with horse serum and/or MGS.

TABLE 10
Round 9 testing of media variables.

| Formulation | Growth |
|---|---|
| Friis + 10% Swine Serum at pH 7.6 | Control |
| AF Friis + 10% Swine Serum | Similar to control |
| AF Friis + 10% Horse Serum + MGS | Poor growth |
| AF Friis + 15% Horse Serum + MGS | Poor growth |
| AF Friis + 10% Horse Serum + MGS | Poor growth |
| (supplemented daily) | |
| AF Friis + 10% Horse Serum + | Poor growth |
| MGS + Yeast Extract | |

A conclusion that may be drawn from this round of variable testing is that AF Friis medium supports Mhp growth similar to control medium in the presence of swine serum, but unlike regular Friis medium, AF Friis medium does not support Mhp growth with horse serum, MGS, and/or yeast extract.

### Example 5

The objective of this study is to confirm potential media formulations identified in Example 4 with a full-scale CCU assay in three independent experiments.

To prepare the inoculum for these experiments, 100 mL of Friis containing 10% swine serum is inoculated in a 500 mL baffled flask with 2×1 mL frozen stock. For each experimental media formulation, 25 mL cultures (final volume) are seeded with 20% (i.e. 5 mL) of a 3 day old culture. Mhp are allowed to grow for three days and then growth is assessed using a full-scale CCU assay, with three independent experimental replications. The full-scale CCU assay is conducted in the same manner as the truncated assay (Example 4) except that in the full-scale assay the Mhp in the sample dilutions are allowed to grow for 14-15 days at 37° C. The cultures are also subjected to sterility (i.e contamination) testing on blood agar at the end of day 3 of culture.

Eighteen different media formulations are tested:

Formulation 1: Friis+10% Swine Serum+MGS

Formulation 2: modified-Porcine-BHI+10% Swine Serum+MGS

Formulation 3: Friis+10% Swine Serum

Formulation 4: modified-Porcine-BHI+10% Swine Serum

Formulation 5: Friis+10% Horse Serum+MGS

Formulation 6: Friis+10% Horse Serum

Formulation 7: Frey+10% Horse Serum+MGS

Formulation 8: modified-Porcine-BHI+10% Horse Serum

Formulation 9: Friis+MGS

Formulation 10: Frey+MGS

Formulation 11: modified-Porcine-BHI+10% Horse Serum+MGS

Formulation 12: Frey+10% Swine Serum

Formulation 13: modified-Porcine-BHI+MGS

Formulation 14: Frey+10% Horse Serum

Formulation 15: Acutone+10% Swine Serum+MGS

Formulation 16: Friis

Formulation 17: Acutone+10% Horse Serum+MGS

Formulation 18: Acutone+MGS

In these experiments, the porcine brain/heart infusion (Porcine-BHI) is modified from the manufacturer's (Becton Dickinson, cat. #BD256120) instructions as shown in Table 11.

TABLE 11
Modification of BD BACTO dehydrated
culture media (Porcine-BHI).

| | BD | As |
|---|---|---|
| | recommendation | modified |
| Component | (g/L) | (g/L) |
| Pork brain | 7.70 | 4.37 |
| Infusion from 250 g | 9.80 | 5.56 |
| pork heart | | |
| No. 2 pork peptone | 10.00 | 5.68 |
| Dextrose | 2.00 | 1.14 |
| Sodium Chloride | 5.00 | 2.84 |
| Disodium Phosphate | 2.50 | 1.42 |
| total | 37.0 | 21.0 |

Both versions contain 0.01 g/L Phenol red.

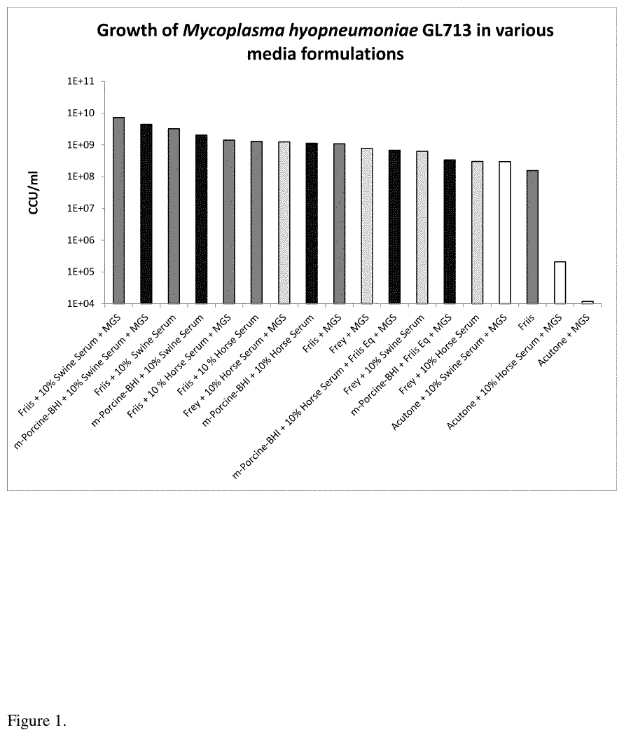

As shown in FIG. 1, except for formulation 17 (Acutone+10% Horse Serum+MGS) and formulation 18 (Acutone+MGS), the formulations supported Mhp growth comparable to that of the control formulation, Friis plus swine serum. Other media formulations tested but not shown in Figure include two more animal-origin-free base media from Becton Dickinson, AF Friis and AF PPLO. These media also did not support Mhp growth in the absence of swine serum. It is interesting that Friis medium without any serum or MGS supported Mhp growth fairly well; however, Friis contains bovine brain infusion, which is not acceptable for global product registration. Also not shown in FIG. 1, Mhp grows poorly in m-BHI and Frey without any serum or MGS.

Of the 16 media formulations that supported Mhp growth, five media formulations—Friis+10% Swine Serum (control, Formulation 3), m-P-BHI+10% Horse Serum+MGS (Formulation 11), m-P-BHI+10% Horse Serum+MGS (supplemented daily), m-P-BHI+MGS (Formulation 13), Frey+10% Horse Serum+MGS (Formulation 7) and Frey+MGS (Formulation 10)—were used for further validation in a large scale (as opposed to flask-scale) fermenter system.

### Example 6

The objective of this study is to compare medium formulations selected from flask studies (Example 5) in a large-scale fermenter system (Ambr 250, Sartorius), which more closely mimics commercial manufacturing conditions. The variables tested in these experiments are:
1. Base medium: Friis (control), Frey, and modified-Porcine Brain Heart Infusion (m-P-BHI);
2. Serum: 10% swine serum (control), 10% horse serum, or no serum; and
3. MGS: supplemented daily or only upon initial culture.

To prepare the inoculum for this experiment, a 25 mL culture is inoculated with 0.5 mL working stock which was grown for three days. Twenty mL of this “pre-inoculum” is used to inoculate 200 mL of Friis+10% swine serum with FoamAway antifoam (Gibco, 0.75 ml/L). This inoculum is incubated at 37° C. with 100 RPM orbital shaking for three days prior to being used to inoculate the fermenters at a 1% (v/v) rate.

Six varied medium formulations are tested:

Formulation 1: Friis+10% Swine Serum (control);

Formulation 2: m-P-BHI+10% Horse Serum+MGS;

Formulation 3: m-P-BHI+10% Horse Serum+MGS (supplemented daily);

Formulation 4: m-P-BHI+MGS;

Formulation 5: Frey+10% Horse Serum+MGS; and

Formulation 6: Frey+MGS.

Mhp is cultured for four days in each of the six formulations. Full-scale CCU assays are performed daily. At the end of day 3, aliquots are taken and subjected to sterility (i.e. contamination) testing on blood agar. At the end of the four-day culture, the Mhp is collected for proteomics analysis as described in Example 7.

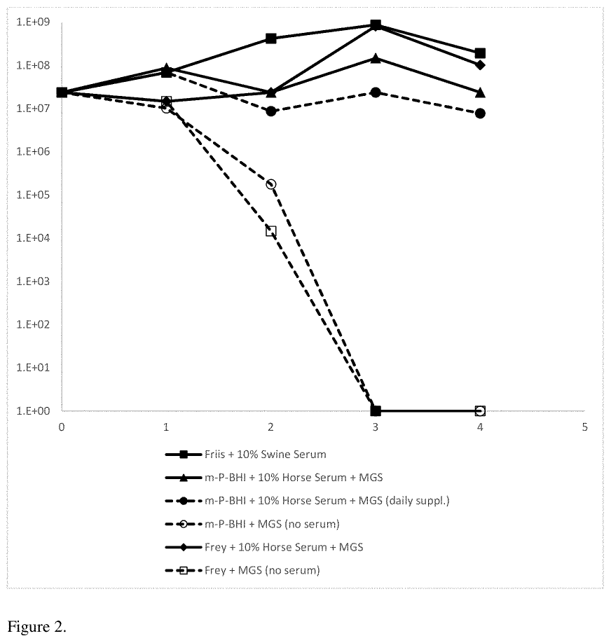

As shown in FIG. 2, Mhp growth in Frey+10% Horse Serum+MGS (Formulation 5), m-P-BHI+10% Horse Serum+MGS (Formulation 2), and m-P-BHI+10% Horse Serum+MGS (supplemented daily)(Formulation 3) is very similar to that of the control Formulation 1, Friis+10% Swine Serum. However, Mhp growth in m-P-BHI+MGS and Frey+MGS is poor compared to the control medium. One possible explanation for the difference with the flask-scale studies in Example 5 is that the fermenter studies are done with a very low inoculum of 1% to discern an extreme effect of the selected media formulations on Mhp growth.

### Example 7

The objective of this study is to determine if the absence of swine serum and/or animal origin ingredients in the selected media formulations significantly alter the global protein profile of Mhp, which could decrease the antigenic properties of the cells and diminish the immunogenic effect of the vaccine product.

Approximately 80 mL is collected from the fermenter cultures described in Example 6 and centrifuged at 10,000 rpm for 30 minutes at 4° C. The cell pellets are washed once in 1 mL of ice-cold PBS and then lysed in 8M urea, 150 mM NaCl, 50 mM Tris-Cl, pH 8.0 for 1 hour. The cell lysate is submitted to MSBioWorks (Ann Arbor, Mich., USA) for proteomics analysis.

The protein expression profiles of Mhp grown in selected media formulations are very similar to that of Mhp grown in Friis plus swine serum (control). These data suggest that the absence of swine serum in the selected media formulations do not appear to alter the protein expression profile of Mhp. Mhp grown in selected media formulations may retain similar immunogenicity to that of Mhp grown in Friis plus swine serum, but this is to be experimentally confirmed. The composition of final media formulations that will be used to grow Mhp for in vivo immunogenicity efficacy are described in Table 12.

TABLE 12
Mycoplasma media formulations.

| Frey base (BBL Mycoplasma Broth) | | Porcine-BHI (BD BACTO, modified) | |
|---|---|---|---|
| Becton Dickinson cat. #212346 | | Becton Dickinson cat. #BD256120 | |
| component | g/L | component | g/L |
| Pancreatic digest of casein | 7.50 | Pork brain | 4.37 |
| Papaic digest of | 2.50 | Infusion from 250 g | 5.56 |
| soybean meal | | pork heart | |
| Yeast extract | 5.00 | No. 2 pork peptone | 5.68 |
| Potassium chloride | 0.40 | Dextrose | 1.14 |
| Sodium Chloride | 5.00 | Sodium Chloride | 2.84 |
| Disodium Phosphate | 1.60 | Disodium Phosphate | 1.42 |
| Monopotassium phosphate | 0.10 | | |
| Magnesium sulfate | 0.20 | | |
| Total suggested use rate | 21.0 | Total modified use rate | 21.0 |

Both basal media also contain 0.01 g/L Phenol red. Complete media include up to 10% heat-inactivated horse serum (HS) and 1X MGS (Table 1). MGS should be prepared as 100X solution and stored at −20° C. in aliquots for further use. Once thawed, MGS should not be frozen for reuse.

### Example 8

The objective of this study is to evaluate the efficacy of Mhp fractions of combinatorial Mhp and Porcine Circovirus Type 2 (PCV-2) vaccines when the Mhp (X+3 stock) is grown in the final media formulations. The vaccination-challenge experiment is a controlled, randomized and single-blinded study.

TABLE 13
Vaccination/challenge study design.

| day | Event |
|---|---|
| ≤−5 | Pigs 21-28 days old arrive and placed into treatment groups. All groups (with |
| | the exception of Group 7) are administered EXCEDE at arrival. All pigs are |
| | screened and confirmed to be negative for Mhp and PCV-2 infection or exposure. |
| −1 | Clinical observations and rectal temperatures collected on all pigs once daily. |
| | Individual body weights collected on all pigs. |
| 0 | Clinical observations and rectal temperatures collected on all pigs once daily. |
| | Collect 2 × 10 mL blood collected from all pigs into serum separator tubes |
| | (SSTs). Administer experimental and control vaccines according to Table 14. |
| 27 | Clinical observations and rectal temperatures collected on all pigs once daily. |
| 28 | Clinical observations and rectal temperatures collected on all pigs once daily. |
| | Collect 2 × 10 mL SSTs from all pigs. |
| | Challenge all pigs according to Table 14. |
| 29 | Clinical observations collected on all pigs once daily. |
| | Challenge all pigs according to Table 14. |
| 30-55 | Clinical observations collected on all pigs once daily. |
| 56 | Clinical observations collected on all pigs once daily. |
| | Collect 2 × 10 mL SSTs from all pigs. |
| | All pigs individually weighed. |
| | All pigs euthanized, necropsied, lungs scored, and lung tissue (fresh and fixed) collected. |

TABLE 14
Treatment groups.

| Group | Treatment | Adjuvant | Route¹ | Dose (mL) | No. pigs |
|---|---|---|---|---|---|
| 1 | PCV-2 only | 50-50 W/O/W² | IM | 1.0 | 20 |
| 2 | Mhp (grown in p-BHI/HS/MGS) + PCV-2 | 50-50 W/O/W | IM | 1.0 | 20 |
| 3 | Mhp (grown in p-BHI/HS/MGS) + PCV-2 | 50-50 W/O/W | ID | 0.2 | 20 |
| 4 | Mhp (grown in Frey/HS/MGS) + PCV-2 | 50-50 W/O/W | IM | 1.0 | 20 |
| 5 | Mhp (grown in Frey/HS/MGS) + PCV-2 | 50-50 W/O/W | ID | 0.2 | 20 |
| 6 | FOSTERA Mhp + PCV-2 | METASIM | IM | 2.0 | 20 |
| 7³ | controls | none | none | none | 6 |

¹Route of administration is either intradermal (ID) or intramuscular (IM).
²Antigen mixed 1:1 (i.e. 50% each) with water/oil in water adjuvant.
³Group 7 consists of sentinel animals sacrificed on the day of arrival to confirm the study animals do not have pre-existing lung lesions.

To prepare the experimental vaccines, PCV2 viral-like particles are prepared using a baculovirus expression system in Sf9 MCS cells (insect cell line). The supernatant containing the PCV2 antigen (50 μg/ml final concentration) is mixed with diluent control or inactivated Mhp, followed by the addition of a W/O/W formulation at a 50%-50% final concentration.

An experimental Mhp vaccine is deemed to be efficacious if: (1) reduction in the treatment group mean lung consolidation is >40% compared to the negative control (Group 1), (2) the mitigated fraction in the vaccinates is >0.35, and (3) the lower 95% confidence interval of the mitigated fraction is >0.2. In 3-4 week old pigs, post challenge lung consolidation in the negative control animals is anticipated to be 8-15% (group mean response) of the lung. A positive vaccine impact equates to a reduction in the group mean response of the vaccinates compared to the controls. The reduction of the lung lesions in vaccinates should be at least 40%. The data set is tested for statistical significance (two-sided p<0.05) by comparing the percent lung consolidation recorded from animals in the vaccinated group to the percent lung consolidation recorded from animals in the non-vaccinated (negative control) group subsequent to a virulent Mhp challenge. Challenge material is GL713 grown in control media. Mitigated fraction and the associated 95% lower confidence bound (LCB) for the investigational vaccine groups (Groups 1-5) against the control groups (Group 7) is estimated for percent lung consolidation. Group 6 is included as a positive control group.

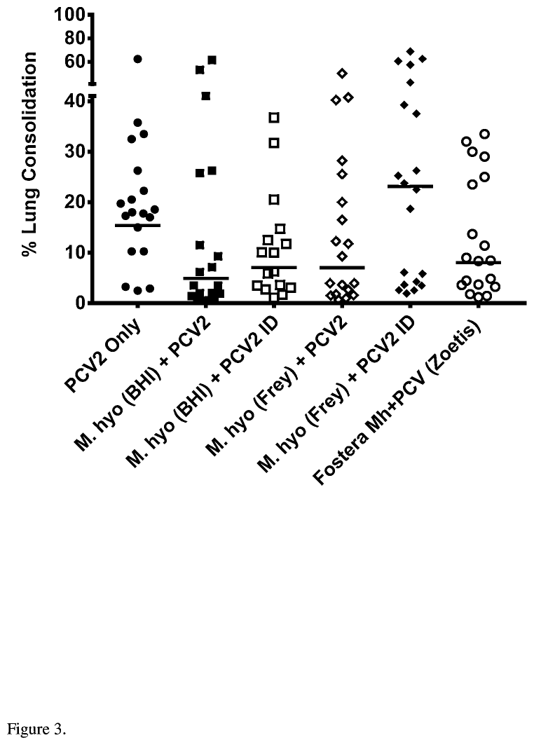

The primary outcome of the experiment is the amount of lung consolidation in the challenged animals as examined at necropsy and recorded as a percentage of the animal's total lung size. As shown in FIG. 3, as expected animals vaccinated only with PCV-2 antigens are not protected from a challenge with virulent Mhp. When Mhp are grown in porcine-BHI+HS+MGS, inactivated by adding 2-bromoethylamine to a final concentration of 4 mM, and formulated into a vaccine, the Mhp antigens provide protection from a challenge with virulent Mhp, regardless of whether the vaccination is administered intradermally or intramuscularly. However, antigens from Mhp grown in Frey base medium+HS+MGS are only effective when administered intramuscularly. The three effective Mhp vaccines demonstrate similar or even better potency than the positive control vaccine.

Daily clinical observations are evaluated and reported as secondary outcomes of the experiment. Table 15. Additionally, injection site lesions and meat quality of the immunization sites of the different experimental vaccines are evaluated at necropsy to establish baseline slaughter withdrawal data.

TABLE 15
Secondary treatment outcomes.

| | | | | | Mean body |
|---|---|---|---|---|---|
| | | | Measurable injection | | weight (lbs) at |
| Group | Treatment | Route | site reactions | mortality | harvest (± SD) |
| 1 | PCV-2 only | IM | 2/20 | 1/19 | 92.6 ± 10.2 |
| 2 | Mhp (p-BHI) + PCV-2 | IM | 0/20 | 1/19 | 98.6 ± 9.6 |
| 3 | Mhp (p-BHI) + PCV-2 | ID | 0/20 | 1/16 | 96.9 ± 14.3 |
| 4 | Mhp (Frey) + PCV-2 | IM | 1/20 | 1/19 | 102.0 ± 12.7 |
| 5 | Mhp (Frey) + PCV-2 | ID | 0/20 | 1/20 | 101.6 ± 13.2 |
| 6 | FOSTERA Mhp + PCV-2 | IM | 1/20 | 1/19 | 93.3 ± 14.7 |

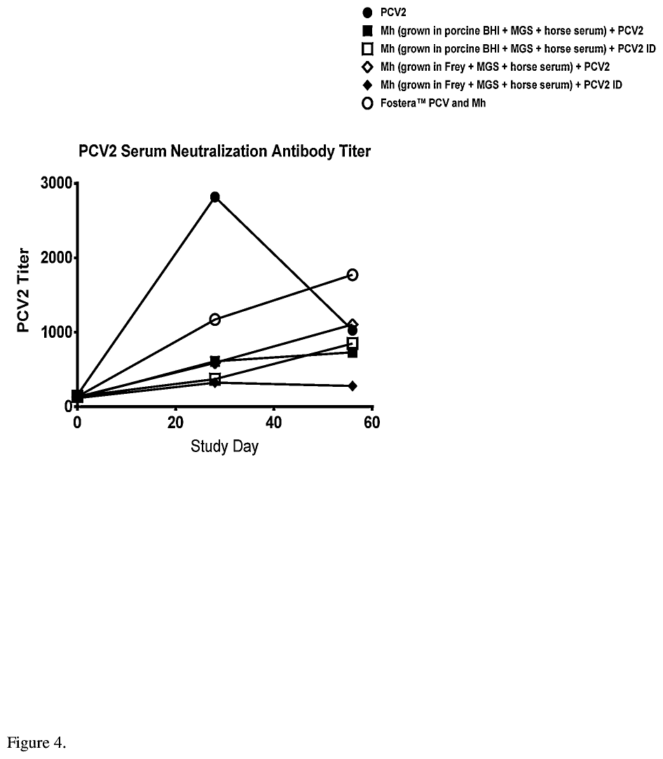

A final study variable is to determine whether the Mhp antigenic fraction, when grown in the experimental media formulations, interferes with the immunogenicity of the PCV-2 antigen when the Mhp and PCV-2 antigens are combined. As shown in FIG. 4, all experimental vaccines are able to elicit anti-PCV2 antibodies when administered to swine, although intradermally-administered Mhp (Frey)+PCV2 again performed suboptimally. Vaccinates from Groups 2-4 respond in a comparable manner to the PCV2 control, Group 1.

SEQUENCE LISTING
Mhyo-PCR-165-rRNA Forward primer
SEQ ID NO: 1
5′ GTAGAAAGGAGGTGTTCCATCC 3′
Mhyo-PCR-16S-rRNA Reverse primer
SEQ ID NO: 2
5′ ACGCTAGCTGTGTGCTTAAT 3′

1. A composition comprising choline chloride, niacinamide, nicotinic acid, L-methionine, L-cysteine, putrescine dihydrochloride, thiamine pyrophosphate, sodium L-ascorbate, spermine, pyridoxal 5′-phosphate monohydrate, tetrahydrofolic acid, 3′-dephosphocoenzyme, and riboflavin;
wherein the composition is a Mycoplasma growth supplement (“MGS”).

2. The composition of claim 1, wherein the Mycoplasma is selected from the group consisting of M. hyosynoviae; M. suis; M. hyorhinitis; and M. hyopneumoniae (“Mhp”).

3. The composition of claim 1, wherein the Mycoplasma is M. hyopneumoniae.

4. A method of culturing Mycoplasma, comprising placing Mycoplasma in a media comprising:
basal medium selected from the group consisting of Frey's medium and porcine brain heart infusion (p-BHI) medium;
horse serum; and
a Mycoplasma growth supplement.

5. The method of claim 4, wherein the Mycoplasma is selected from the group consisting of M. hyosynoviae; M. suis; M. hyorhinitis; and M. hyopneumoniae (“Mhp”).

6. The method of claim 4, wherein the Mycoplasma is M. hyopneumoniae.

7. The method of claim 4, wherein the horse serum is present at about 2.5% to about 10% v/v.

8. The method of claim 4, wherein the horse serum is present at about 5% to about 10% v/v.

9. The method of claim 4, wherein the horse serum is present at about 10% v/v.

10. The method of claim 4, wherein components of the MGS are present at final concentrations of:
about 0.5 mg/L choline chloride;
about 0.025 mg/L niacinamide;
about 0.025 mg/L nicotinic acid;
about 0.1 mM L-methionine;
about 1.5 mM L-cysteine;
about 0.1 mM putrescine dihydrochloride;
about 0.01 mg/L thiamine pyrophosphate;
about 0.284 mM sodium L-ascorbate;
about 0.1 mM spermine;
about 0.025 mg/L pyridoxal 5′-phosphate monohydrate;
about 0.05 mg/L tetrahydrofolic acid;
about 0.025 mg/L 3′-dephosphocoenzyme A; and
about 0.01 mg/L riboflavin.

11. The method of claim 4, wherein the Mycoplasma is cultured at 37° C. for 3-15 days.

12. A method of preparing an immunogenic composition comprising:
culturing Mycoplasma in a medium comprising basal medium selected from the group consisting of Frey's medium and porcine brain heart infusion (p-BHI) medium; horse serum; and a Mycoplasma growth supplement;
incubating the Mycoplasma at 37° C.; and
inactivating the Mycoplasma.

13. The method of claim 12, wherein inactivating the Mycoplasma is performed with 2-bromoethylamine.

14. A product comprising a composition of claim 1 for a medicament for preventing, reducing, or ameliorating diseases caused by Mycoplasma.

15. The product of claim 14, where the medicament is for treatment of swine.
